(19) United States
(12) Patent Application Publication
(10) Pub. No.: US 2026/0283450 A1
(43) Pub. Date: Sep. 24, 2026

(54) VALVED ANUS SEAL AND METHOD OF USE FOR COLONOSCOPY

(71) Applicant: Mayo Foundation for Medical Education and Research, Rochester, MN (US)

(72) Inventors: Jana G. Al Hashash, Jacksonville, FL (US); Johanna L. Chan, Ponte Vedra Beach, FL (US); Shawn Robinson, Fleming Island, FL (US)

(21) Appl. No.: 19/562,012

(22) Filed: Mar. 10, 2026

Related U.S. Application Data
(60) Provisional application No. 63/774,469, filed on Mar. 19, 2025.

(51) Int. Cl. A61B 1/00

(52) U.S. Cl. CPC A61B 1/00154; A61B 1/00131

(57) ABSTRACT

Devices and methods for use with colonoscopy procedures. Embodiments of the device include a diametrically-expandable seal member configured to be interested into an anus of a patient, and a valve coupled to the seal member. The seal member defines a lumen extending through the seal member that is configured to allow a colonoscope to extend through the seal member and into the patient's bowel. The valve is configured to allow the colonoscope to extend through the seal member and to inhibit insufflation gas from escaping the patient's bowel through the lumen. Embodiments of the method include inserting the colonoscope through the valve of the seal member, inserting the colonoscope and the seal member into the patient's anus, and insufflating the patient's bowel with gas through the colonoscope.

### CROSS-REFERENCE TO RELATED APPLICATION

This application claims the benefit of Provisional Application No. 63/774,469, filed Mar. 19, 2026, which is incorporated herein by reference in its entirety for all purposes.

### TECHNICAL FIELD

This disclosure relates generally to devices and methods for use in connection with colonoscopy procedures.

### BACKGROUND

Colonoscopy procedures are commonly performed for purposes such as colorectal cancer screening, diagnosis and monitoring of inflammatory bowel diseases, evaluation of rectal bleeding and diarrhea, and other gastrointestinal (GI) indications. Colonoscopy is typically performed using a colonoscope, an instrument that includes an elongated and flexible member (e.g., about 13 mm in diameter for devices used in adults) that is inserted the anus of the patient. The distal end of the colonoscope includes a camera that is used to view the inside of the patient's colon as the colonoscope is advanced through and/or removed from the bowel. Colonoscopes typically also include a number of lumens and ports to provide other functions during the procedures. For example, a lumen and port may provide access for instruments used to remove and collect polyps or other tissue, and/or to provide gas used to insufflate the colon.

Insufflation, the insertion of gas into the colon to expand the colon during the procedure, is important for the examination of the colonic mucosa in its entirety and to reduce the possibility that colonic lesions are missed and to ensure that other lesions are treated. Obtaining adequate insufflation is sometimes difficult, for example when patients are unable to hold the gas in the colon due to a lax anal sphincter, a condition that may also be compounded when the sphincters are further relaxed because of anesthesia. The inability to fully or adequately insufflate the colon may compromise the quality of the examination, may increase the risk that colonic lesions are missed and not adequately treated, and may impact the ability to perform therapeutic maneuvers such as hemostasis or polypectomy. The inability to fully or adequately insufflate the colon may also result in longer procedure times, for example as the endoscopist tries different maneuvers to examine otherwise collapsed mucosa.

Known approaches for preventing gas from escaping from the colon include the manual application of gluteal cheek pressure to physically occlude the anal sphincter. This approach may provide variable success rates, and may not be effective on certain patients such as those that are thin and lack gluteal adipose tissue, or in patients with anal-rectal anatomic abnormalities. The application of gluteal cheek pressure may also divert assisting personnel from other procedures.

There remains a continuing need for improved devices and methods for use in connection with colonoscopy. Such devices and methods that can effectively maintain colon insufflation would be advantageous. Such devices and methods that can be relatively easily and conveniently be used and performed, would be especially desirable.

### SUMMARY

Disclosed embodiments include anal seal devices that can be used in connection with colonoscopy to enhance insufflation during the procedures. The devices can be relatively easily used, and can enhance the efficacy of colonoscopy procedures.

One example is a device comprising a seal member and a valve. The seal member is configured to be inserted into an anus of a patient and includes a proximal end and a distal end. The seal member defines a lumen extending through the seal member between the proximal end and the distal end, and that is configured to allow a colonoscope to extend through the seal member and into a patient's bowel. The valve is coupled to the seal member and configured allow the colonoscope to extend through the seal member and to inhibit insufflation gas from escaping the patient's bowl through the lumen.

In some embodiments, the seal member is a diametrically-expandable member. For example, the seal member may be a self-expandable member. At least portions of the seal member may be configured to compress toward a reduced-diameter configuration when the device is inserted into the patient's anus. Some embodiments further include a constraining member movable between a delivery position and an operative position with respect to the seal member, wherein: when the constraining member is in the delivery position the constraining member constrains the seal member to a reduced-diameter delivery configuration; and when the constraining member is in the operative position, the seal member expands.

In some embodiments the seal member comprises an expandable balloon.

In some embodiments the seal member is configured to engage at least portions of the patient's anal canal. For example, at least portions of the seal member may be configured to be located in the patient's rectum.

Some embodiments further comprise a hub including a tubular portion including a proximal end and a distal end extending through at least portions of the lumen of the seal member. For example, the valve may be coupled to the hub. The hub may include a base portion that extends from the proximal end of the seal member. At least portions of the valve may be coupled to the base portion of the hub.

In some embodiments, at least portions of the tubular portion of the hub include an inner diameter tapering in a reducing-diameter configuration with increasing distance from the proximal end. For example, the tubular portion of the hub may include one or more longitudinally-extending slots. At least portions of the tubular portion of the hub may include an outer diameter tapering in a reducing-diameter configuration with increasing distance from the proximal end; and at least portions of the lumen of the seal member may include an inner diameter tapering in a reducing-diameter configuration with increasing distance from the proximal end, and the at least portions of the lumen of the seal member mate with the at least portions of the tubular portion of the hub.

In some embodiments, the distal end of the tubular portion of the hub does not extend to the distal end of the seal member.

In some embodiments, the seal member comprises a distal portion including an outer diameter that tapers between a first diameter and a second diameter that is less than the first diameter with increasing distance from the proximal end. For example, the seal member may comprise a proximal portion including an outer diameter that tapers between a third diameter that is greater than the first diameter and a fourth diameter that is less than the first diameter with increasing distance from the proximal end.

In some embodiments, the device is configured to open along its length to allow the device to be inserted from its side onto a colonoscope.

Another example is a method for performing colonography. The method may comprise inserting a colonoscope through a valve in a lumen of an anal seal member; inserting the colonoscope into an anus of a patient; and insufflating the patient's bowel with gas through the colonoscope. The seal member may, for example, comprise a seal member in accordance with any or all embodiments of the example described above.

Some embodiments further comprise releasably attaching the anal seal member to a portion of the colonoscope not inserted into the patient's anus. For example, the method may further comprise moving the anal seal member along the colonoscope from its releasably attached position, and inserting the anal seal member in the patient's anus; and insufflating the patient's bowel with gas when the anal seal member is inserted in the patient's bowel.

Some embodiments further comprise inserting the anal seal member in the patient's anus; and insufflating the patient's bowel with gas when the anal seal member is inserted in the patient's bowel.

While the disclosure is amenable to various modifications and alternative forms, specific embodiments have been shown by way of example in the drawings and are described in detail below. The disclosure, however, is not limited to the particular embodiments described. On the contrary, the disclosure is intended to cover all modifications, equivalents, and alternatives falling within the scope of the disclosure as defined by the appended claims.

### BRIEF DESCRIPTION OF THE DRAWINGS

FIG. 1 is a diagrammatic isometric view of an anus seal device, in accordance with embodiments.

FIG. 2 is a side view of the anus seal device shown in FIG. 1, in accordance with embodiments.

FIG. 3 is a top view of the anus seal device shown in FIG. 1, in accordance with embodiments.

FIG. 4 is a sectional view of the anus seal device shown in FIG. 1, in accordance with embodiments.

FIG. 5 is an exploded isometric view of the anus seal device shown in FIG. 1, in accordance with embodiments.

FIG. 6 is an illustration of a colonoscope, and an anus seal device in accordance with embodiments, being mounted onto a colonoscope.

FIG. 7 is a diagrammatic illustration of a colonoscope inserted into a bowel of a patient though the patient's anus.

FIG. 8 is a diagrammatic illustration of a colonoscope inserted in a patient's bowel, and an anus seal device in accordance with embodiments being moved toward a distal end of the colonoscope and inserted into the patient through the patient's anus.

FIG. 9 is a diagrammatic illustration of a colonoscope and an anus seal device such as that shown in FIG. 1 in accordance with embodiments, inserted into a patient through the patient's anus.

FIG. 10 is a diagrammatic isometric view of an anus seal device, in accordance with embodiments.

FIGS. 11A and 11B are diagrammatic side view illustrations of an anus seal device in accordance with embodiments, where FIG. 11A shows the device in an expanded-diameter, operative configuration, and FIG. 11B shows the device in a reduced-diameter, delivery configuration.

FIG. 12 is a diagrammatic illustration of a colonoscope and an anus seal device such as that shown in FIGS. 11A and 11B in accordance with embodiments, inserted into a patient thru the patient's anus.

FIGS. 13A and 13B are isometric views of an anus seal device in accordance with embodiments, where FIG. 13A shows the device in an expanded-diameter, operative configuration, and FIG. 13B shows the device in a reduced-diameter, delivery configuration.

FIG. 14 is a top view of the anus seal device shown in FIGS. 13A and 13B.

FIG. 15 is a diagrammatic illustration of a colonoscope and an anus seal device such as that shown in FIGS. 13A and 13B in accordance with embodiments, inserted into a patient thru the patient's anus.

FIG. 16 is a diagrammatic isometric illustration of an anus seal device, in accordance with embodiments.

FIG. 17 is a diagrammatic illustration of a colonoscope and anus seal device such as that shown in FIG. 16 in accordance with embodiments, mounted to a patient.

FIG. 18 is a diagrammatic isometric illustration of an anus seal device, in accordance with embodiments.

FIG. 19 is a diagrammatic illustration of a colonoscope and anus seal device such as that shown in FIG. 18 in accordance with embodiments, mounted to a patient.

FIG. 20 is a diagrammatic illustration of a colonoscope and anus seal device, in accordance with embodiments, mounted to a patient.

While the disclosure is amenable to various modifications and alternative forms, specific embodiments have been shown by way of example in the drawings and are described in detail below. The disclosure, however, is not limited to the particular embodiments described. On the contrary, the disclosure is intended to cover all modifications, equivalents, and alternatives falling within the scope of the disclosure as defined by the appended claims.

### DETAILED DESCRIPTION

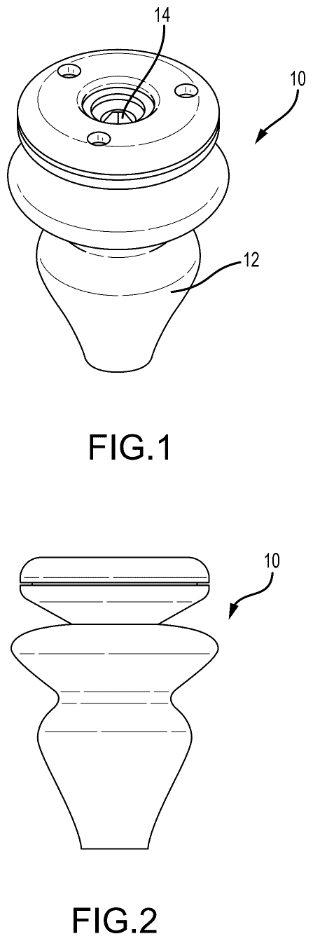

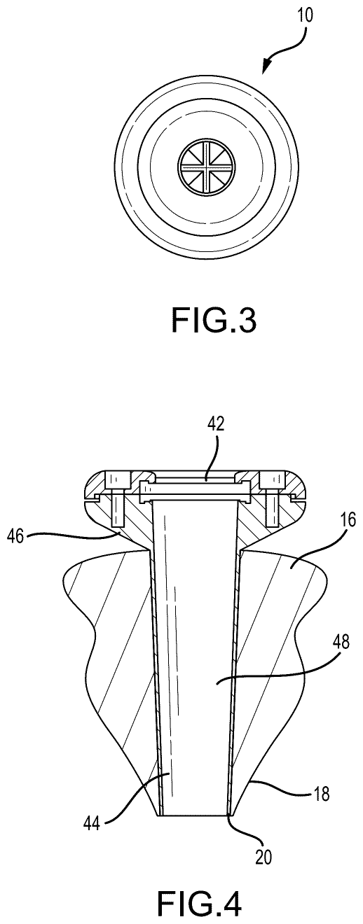

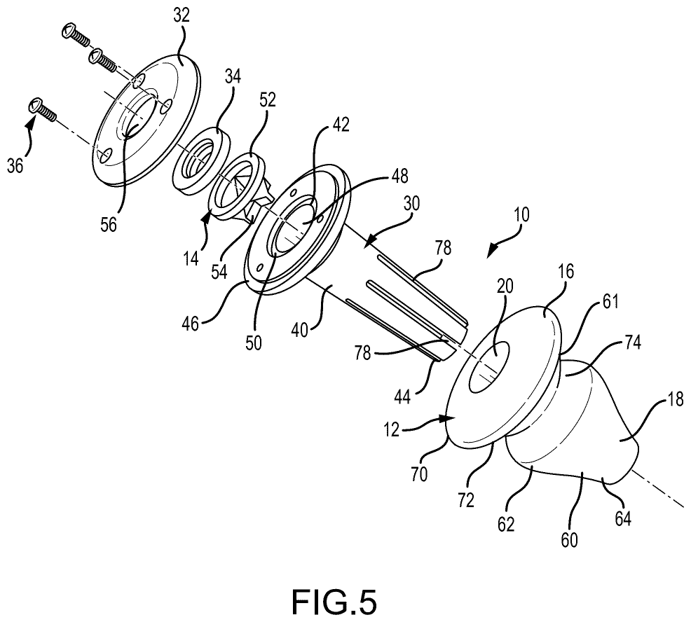

FIGS. 1-5 illustrate an anus seal device 10 configured for use in connection with colonoscopy procedures, in accordance with embodiments. As shown, the device 10 includes a seal member 12 configured to be inserted into an anus of a colonoscopy patient, and a valve 14. The seal member 12 includes a proximal end 16, a distal end 18, and defines a lumen 20 that extends through the seal member between the proximal and distal ends. As shown in greater detail below, the lumen 20 is configured (e.g., sized and/or shaped) to enable or allow a colonoscope to extend through the seal member 12 and into the patient's rectum/bowel during colonoscopy. The colonoscope can be operated to deliver gas into the patient's bowel during colonoscopy to insufflate the bowel and enhance the efficacy of the procedure. Valve 14 is configured to enable or allow the colonoscope to extend through the seal member 12, and to prevent or inhibit the insufflation gas from escaping the bowel through the lumen 20 during the procedure. For example, the valve 14 can be a one-way valve that allows a colonoscopist to move and manipulate the colonoscope through the seal member 12 and the valve during the procedure (e.g., to advance and withdraw the colonoscope), while inhibiting the flow of insufflation gas from the bowel through the seal member and valve.

The illustrated embodiments of the anus seal device 10 also include a hub 30, a hub top 32, washer 34 and fasteners such as screws 36. In these embodiments the hub 30 includes a tubular portion 40 having a proximal end 42 and a distal end 44, and a base portion 46 on the proximal end. The tubular portion 40 defines a lumen 48 that extends through the hub 30 between the proximal end 42 and distal end 44. The base portion 46, which has a larger diameter than the tubular portion 40, may be characterized as a shoulder, and is configured to receive the valve 14 with the valve located in the lumen 48. The illustrated embodiments, for example, show an annular recess 50 in the proximal end portion of the base portion 46 that receives and seats a rim 52 of the valve 14, while other portions of the valve such as the leaflets 54 are located in the lumen 48. The hub top 32 can be attached to the proximal end portion of the base portion 46 by the screws 36 to secure the valve 14 to the base portion, with the washer 34 located between the rim 52 of the valve and the hub top to provide a seal between the valve rim and an opening 56 through the hub top. Valve 14 can be a cross-slit seal type of device such as that commercially available from MiniValve, although other embodiments may include other valves to provide the functionality described herein.

Seal member 12 is a diametrically-expandable member in embodiments, and is shown for example in FIGS. 1-5 in a neutral or uncompressed state or configuration with no external forces applied to the seal member. In embodiments, for example, the seal member 12 can be Flexfoam-it 14, commercially available from Smooth-On. In embodiments, the seal member 12 is sufficiently flexible that at least portions can be compressed to a reduced-diameter configuration before or during (e.g., by) the insertion of the seal member into the patient's anus. Embodiments of the seal member 12 may be self-expandable. For example, after forces applied to the seal member 12 that cause the seal member to move toward the reduced-diameter configuration are removed, and/or when the seal member has been inserted in the anus of a patient and is in a reduced diameter configuration, the self-expandable portions of the seal member are defined by forces that tend to urge the seal member toward its uncompressed state and into engagement with tissues of the patient.

The illustrated embodiments of the seal member 12 include a distal portion 60 and a proximal portion 61. The distal portion 60 tapers from a first outer diameter portion 62 having a first diameter, to a second outer diameter portion 64 at the distal end 18 having a second diameter that is less than the first diameter. The second diameter at the second outer diameter portion 64 is sufficiently small that the distal end 18 can be inserted through the anus of the patient, and sufficiently large that a colonoscope, and the tubular portion 40 of the hub 30 in embodiments, can be inserted through the seal member 12. In embodiments, for example, the second diameter at the distal end 18 of the seal member 12 is about 16 mm. The first diameter of the first outer diameter portion 62 is, in embodiments, sufficiently large that the associated portions of the seal member 12 cannot generally be inserted through the patient's anus (e.g., without undue force that might injure the patient). In embodiments, for example, the first diameter of the first outer diameter portion 62 is about 41.4 mm. The proximal portion 61 tapers from a third outer diameter portion 70 at the proximal end 16 having a third diameter, to a fourth outer diameter portion 72 having a fourth diameter that is less than the third diameter. In embodiments, the third diameter of the third outer diameter portion 70 is larger than the first diameter of the first outer diameter portion 62 of the distal portion 60, and the fourth diameter of the fourth outer diameter portion 72 is less than the first diameter. For example, in embodiments the third diameter is about 55.9 mm, and the fourth diameter is about 30.5 mm. The illustrated embodiments of the seal member 12 thereby define a neck portion 74 between the distal portion 60 and proximal portion 61. Seal member 12 is about 61 mm long in embodiments. Other embodiments of the seal member 12 have other dimensions and/or shapes.

The inner diameter of the tubular portion 40 of the hub 30 is sufficiently large to enable or allow a colonoscope to extend through the lumen 48. In the illustrated embodiments, the inner diameter of the tubular portion 40 tapers between a first diameter at the proximal end 42 and a second diameter at the distal end 44. In embodiments, for example, the first diameter at the proximal end 42 is about 20 mm, and the second diameter at the distal end 44 is about 15 mm. A device 10 having these exemplary dimensions may, for example, be used with a colonoscope have a diameter of about 13 mm. As shown for example in FIG. 4, the inner diameter of the lumen 20 of the seal member 12 is configured to engage and mate with the outer diameter of the tubular portion 40 of hub 30, and may taper between the proximal end 16 and the distal end 18. In embodiments, the tubular portion 40 of the hub 30 extends the entire length of the lumen 20 of the seal member 12. In other embodiments, the tubular portion 40 of the hub 30 do not extend the entire length of the lumen 20 or to the distal end 18 of the seal member 12 (e.g., portions of the lumen 20 adjacent to the distal end 18 of the seal member are free from the hub).

The illustrated embodiments of the tubular portion 40 of the hub 30 include one or more longitudinally-extending slots 78 (a plurality of slots are shown for purposes of example). Slots 78 can provide or enhance flexibility of the hub 30, enabling or allowing the colonoscopist to bend the colonoscope during procedures. In the illustrated embodiments, the diameter of the base portion 46 of the hub 30 is larger than the diameter of the proximal portion 16 of the seal 12, and necks down in diameter with increasing distance toward the distal end 44 of the tubular portion 40. The base portion 46 of the hub 30, and/or at least portions of the seal member 12, may be grasped and held during insertion of the device 10 into a patient's anus.

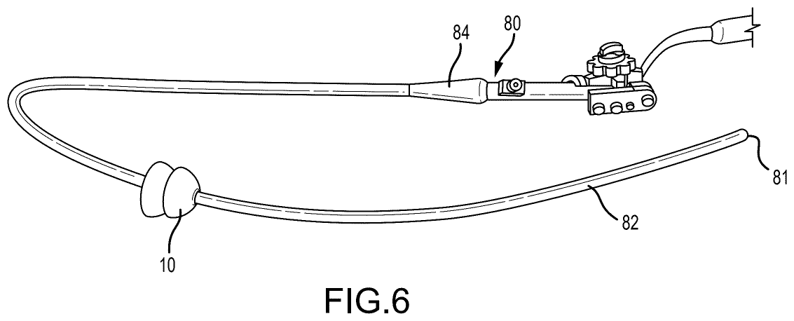

FIGS. 6-9 can be used to describe a method for performing colonoscopy using the anus seal device 10, in accordance with embodiments. As shown for example in FIG. 6, the device 10 can be inserted onto or mounted to a colonoscope 80 by inserting a distal end 81 of the elongated portion 82 of the colonoscope into the opening 56 on the proximal end of the device, through the valve 14 and lumens 20 and 48, and out the distal end of the device (e.g., insertion in a longitudinal or axial direction). The device 10 can then be slid along the elongated portion 82, for example to a position such as that at a proximal portion 84 of the elongated portion, that will reduce or prevent interference with the colonoscopist's insertion of the colonoscope into the patient. In embodiments, the anus seal device 10 can releasably engage the colonoscope at a set-up position prior to the colonoscopy procedures, such as for example by a friction fit with an increasing-diameter portion of the colonoscope 80 at near the proximal portion 84.

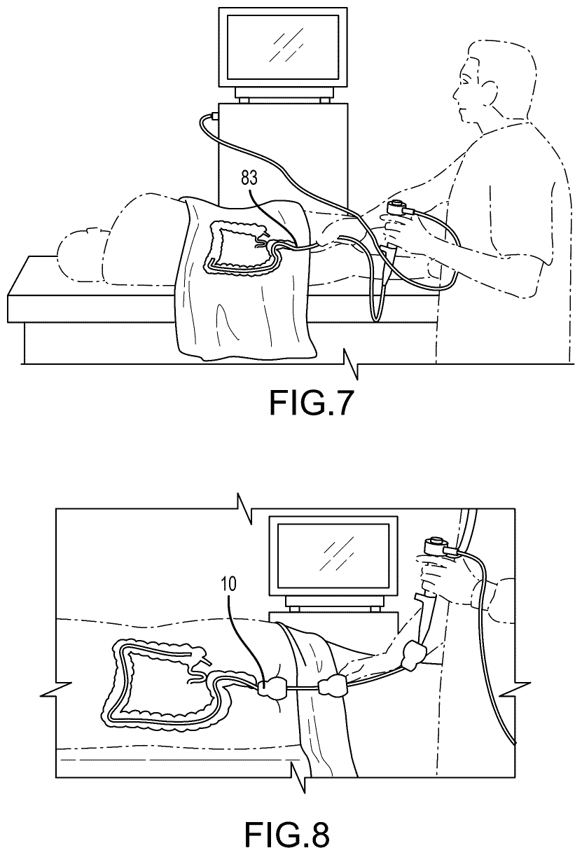

As shown for example in FIG. 7, the colonoscopist may insert the distal end 81 of the colonoscope 80 through the patient's anus 83 and into the bowel, and then actuate the colonoscope to inject gas into the bowel for purposes of insufflation. By some approaches, the colonoscopist may perform the colonoscope insertion and insufflation without inserting the anus seal device 10 into the patient (e.g., the device may remain in its set-up position), and assess whether the patient's colon can be appropriately or adequately insufflated without the use of the anus seal device. If by these approaches it is determined that the use of the anus seal device 10 may enhance the ability to achieve insufflation, the colonoscopist can move the device along the colonoscope (e.g., from the set-up position), and insert it into the patient as shown in FIG. 8. By other approaches the colonoscopist may insert the anus seal device 10 into the patient at the time that the colonoscopist is initially inserting colonoscope and/or insufflating the colon. For example, some colonoscopists may choose to make use of the anus seal device 10 before assessing whether it might enhance insufflation, a decision that could, for example, be made based on the colonoscopists preferences or experience with characteristics of the presenting patient.

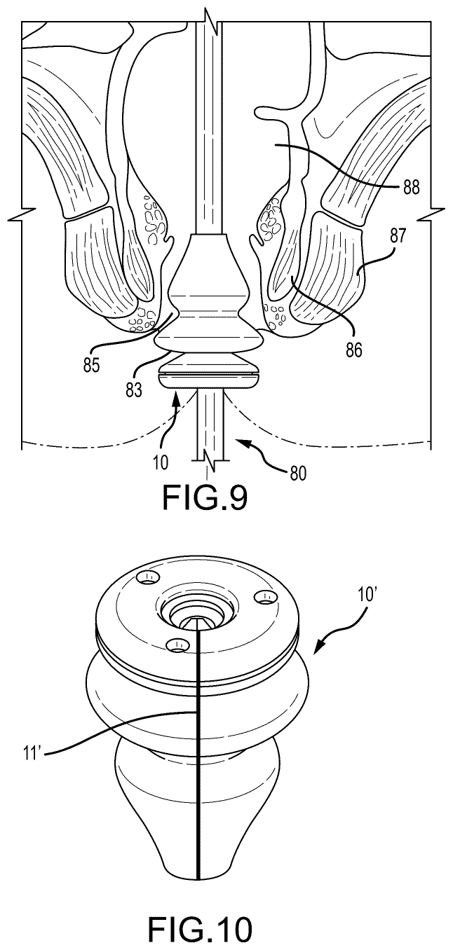

FIG. 9 is an illustration of an anus seal device 10 inserted into the patient in accordance with embodiments. As shown, the anus seal device 10 is located with at least portions of the distal portion 60 in the anal canal 85, and engaging tissue of the patient between at least portions of one or both of the internal anal sphincter 86 and/or the external anal sphincter 87. At least portions of the distal portion 60 of the seal member 12, the proximal portion 61 and the base portion 46 of the hub 30 are also shown outside of the anus 83 in the FIG. 9 embodiment. In other embodiments, the anus seal device 10 may be inserted into the patient's anal canal 85 and/or rectum 88 to greater or lesser distances than shown in FIG. 9 (e.g., while providing the insufflation sealing functionality of the device). The anus seal device 10 may be compressed to a reduced-diameter configuration in the patient by tissues surrounding the anal canal 85 during the insertion procedure. Alternatively or additionally, the anus seal device 10 may be compressed to a reduced-diameter configuration by other approaches, for example by hand, before it is inserted into the patient. By use of the inserted anus seal device 10, the colonoscopist is able to move the colonoscope 80 through the patient's bowl and through the device during colonoscopy, while the escape of insufflation gas is inhibited because of the valve 14.

FIG. 10 is an illustration of an anus seal device 10′ in accordance with embodiments. Anus seal device 10′ can operate and provide the functionality of anus seal device 10 described above, but is configured to be also inserted onto the elongated portion 82 of the colonoscope 80 from the side of the elongated portion (e.g., insertion in a sideways or transverse direction), for example in addition to or alternatively from the approach described above where the device is inserted axially onto the colonoscope. The illustrated embodiments of the anus seal device 10′ include a separation 11′ that extends through the sides of the seal member 12′, hub 30′, valve 14′, washer 34′ and hub top 32′. The seal member 12′, hub 30′, valve 14′, washer 34′ and hub top 32′ are sufficiently flexible and resilient that the device 10′ and these components can be separated at the separation 11′, and pulled sufficiently far apart to enable the elongated portion 82 of the colonoscope 80 to be inserted through the separation and into the lumens of the device, while causing the device and component to be forced back together and close the separation after the colonoscope is inserted into the device. The anus seal device 10′ can then function in the manner described above. In some embodiments, the forces from the patient's body when the device 10′ is inserted, for example forces from the internal sphincter 86 and/or external sphincter 87, may help to close the separation 11′. Yet other embodiments include other configurations that allow anus seal devices to be mounted to the colonoscope from the side of the colonoscope and to provide the functionality described herein. For example, the anus seal device can be formed from two or more structures that can be completely or hingedly separated from one another to provide the opening into the device (e.g., into two components) and then reassembled after they are mounted around the colonoscope.

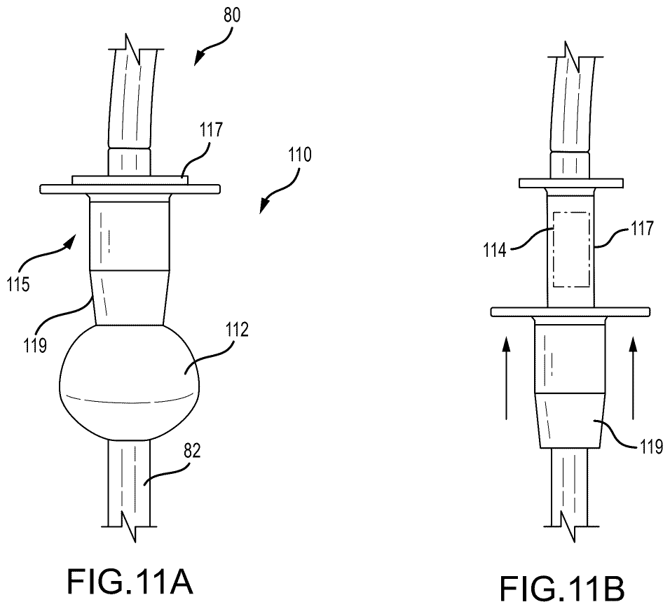

FIGS. 11A and 11B are illustrations of an anus seal device 110 in accordance with embodiments, and are shown mounted to an elongated portion 82 of a colonoscope 80. As shown, the anus seal device 110 includes a self-expandable seal member 112 on an end of an actuator 115. Seal member 112 can be substantially the same as or similar to the seal member 12 of the anus seal device 10 described above. The actuator 115 includes a base member 117 that may be fixedly mounted to the seal member 112, and a constraining member 119 that is moveable (e.g., slidable) with respect to the base member 117. The constraining member 119 is a tubular member in the illustrated embodiments, and has a diameter that is less than a diameter of the seal member 112 in its uncompressed configuration. FIG. 11B shows the anus seal device 110 in a delivery state, with the constraining member 119 moved with respect to the base member 117 over the seal member 112. In the delivery state shown in FIG. 11B, the constraining member 119 constrains or holds the seal member 112 in a reduced-diameter configuration. FIG. 11A shows the anus seal device 110 in an operative state, with the constraining member 119 slid or otherwise moved toward the base member 117 and off of the seal member 112. As shown in FIG. 11A, when the constraining member 119 is off of the seal member 112, the seal member 112 can self-expand toward its unconstrained configuration. A valve 114, which can be substantially the same as or similar to the valve 14 of the anus seal device 10 described above, is shown located in the base member 117 for purposes of example. In other embodiments the valve 114 can be located in other components of the anus seal device 110, such as for example the seal member 112.

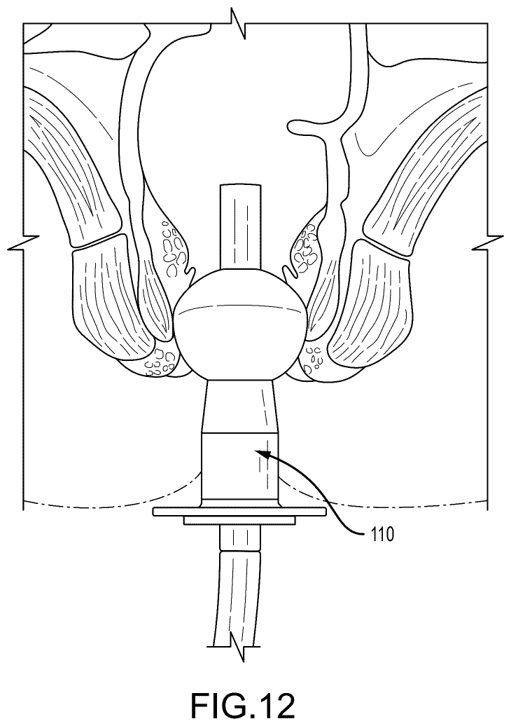

FIG. 12 is an illustration of an anus seal device 110 inserted into the patient in accordance with embodiments. Anus seal device 110 is inserted into the patient through the anus 83 when in the delivery state shown in FIG. 11B, and positioned with the seal member 112 and the over-laying constraining member 119 located beyond the anus. The constraining member 119 may be actuated by sliding it over the base member 117, to allow the seal member 112 to self-expand into a sealing configuration. In the illustrated embodiments, the anus seal device 110 is located with at least portions of the seal member 112 in the anal canal 85, and engaging tissue of the patient between at least portions of one or both of the internal anal sphincter 86 and/or the external anal sphincter 87. At least portions of the seal member 112 are also shown outside of the anus 83 in the FIG. 12 embodiment. In other embodiments the anus seal device 110 may be inserted into the patient anal canal 85 and/or rectum 86 to greater or lesser distances than shown in FIG. 12 (e.g., while providing the insufflation sealing functionality of the device). By use of the inserted anus seal device 110, the colonoscopist is able to move the colonoscope 80 through the patient's bowl and through the device during colonoscopy, while the escape of insufflation gas is inhibited because of the valve 114, in manners similar to those described above.

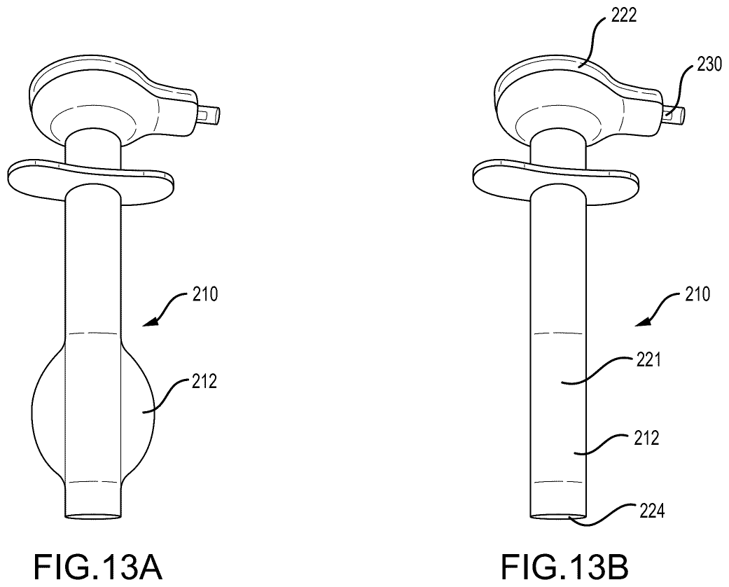

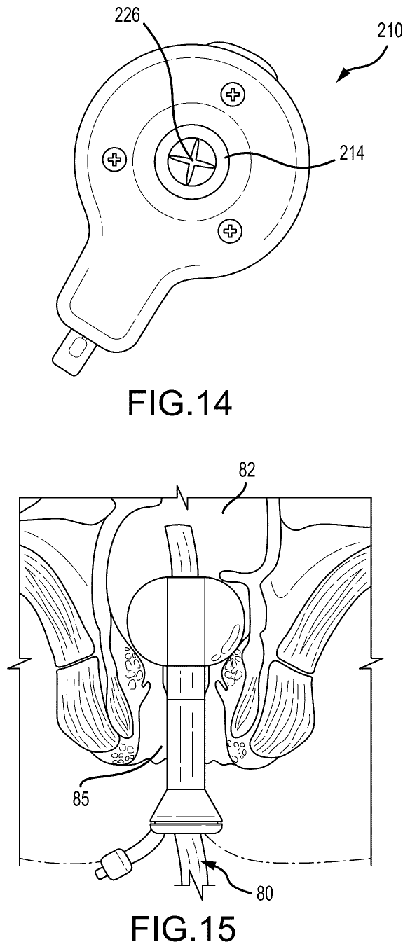

FIGS. 13A, 13B and 14 are illustrations of an anus seal device 210 in accordance with embodiments. As shown, the anus seal device 210 includes a tubular member 221 having a proximal end portion 222 and a distal end portion 224 defining a colonoscope-receiving lumen 226 therethrough. A valve 214 is located in the tubular member 221, and is shown at the proximal end portion 222 in the illustrated embodiments. The valve 214 can be substantially the same as or similar to the valve 14 of the anus seal device 10 described above. In other embodiments the valve 214 can be positioned at other locations of the anus seal device 210, such as for example closer to the distal end portion 224. An inflatable balloon 212 is located on the tubular member 221, and is shown at the distal end portion 224 in the illustrated embodiments. Balloon 212 is coupled to an inflation port 230 by an inflation lumen.

FIG. 13B shows the anus seal device 210 in a delivery state, with the balloon 212 uninflated and in a reduced-diameter, delivery configuration. FIG. 13A shows the anus seal device 210 in an operative state, with the balloon 212 expanded to an enlarged-diameter configuration. Balloon 212 can be inflated by the application of inflation fluid through the inflation port 230.

FIG. 15 is an illustration of an anus seal device 210 inserted into the patient in accordance with embodiments. Anus seal device 210 can be inserted into the patient through the anus 83 when in the reduced-diameter, delivery state shown in FIG. 13B. The balloon 212 can then be inflated to the expanded-diameter configuration and to cause the balloon to engage tissues in the patient. In the illustrated embodiment, the balloon 212 is shown in its expanded-diameter configuration with at least portions located in the patient's rectum and adjacent to the anal canal 85. In other embodiments the anus seal device 210 may be inserted into the patient's anal canal 85 and/or rectum to greater or lesser distances than shown in FIG. 15 (e.g., while providing the insufflation sealing functionality of the device). By use of the inserted anus seal device 210, the colonoscopist is able to move the colonoscope 80 through the patient's bowl and through the device during colonoscopy, while the escape of insufflation gas is inhibited because of the valve 214, in manners similar to those described above.

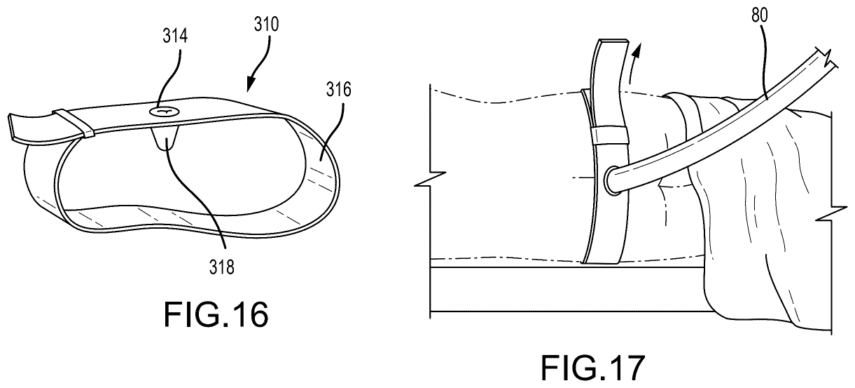

FIGS. 16 and 17 are illustrations of an anus seal device 310 in accordance with embodiments. As shown, anus seal device 310 includes a seal or valve 314 mounted to a cinching device or mechanism such as belt 316. The illustrated embodiments also include a compliant foam member 318 attached to the valve 314. As shown in FIG. 17, the belt 316 can be wrapped around the patient's gluteal cheeks and tightened after the valve 314, and the foam member 318 if present, are inserted into the patient's anus or otherwise positioned to provide access to the patient's bowel through the anus. The valve 314 provides a port through which the elongated portion 82 of the colonoscope 80 can be inserted to the patient's bowel, while also inhibiting the escape of insufflation gas from the bowel. Tightness of the belt can be changed as appropriate or needed to maintain insufflation. In addition to inhibiting the escape of insufflation gas, anus seal device 310 allows the colonoscopist to move the colonoscope 80 through the patient's bowel and through the device during colonoscopy, in manners similar to those described above.

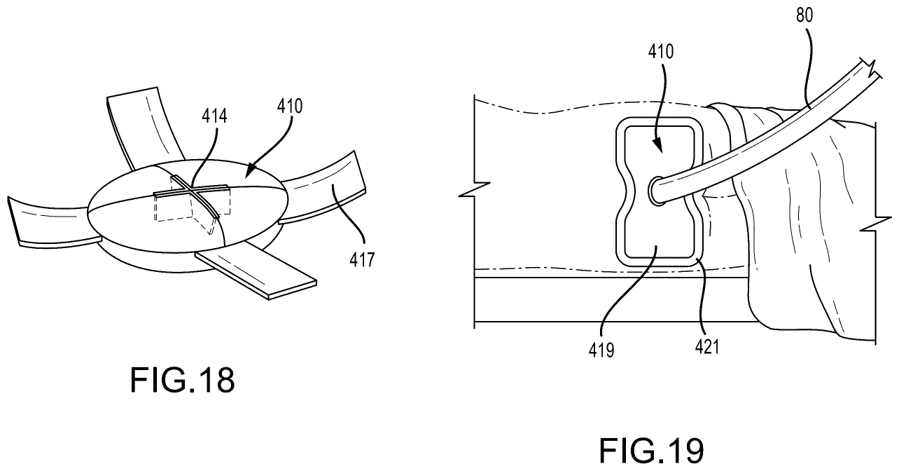

FIGS. 18 and 19 are illustrations of an anus seal device 410 in accordance with embodiments. As shown, anus seal device 410 includes a seal or valve 414 mounted to a base 415, which can, for example, include a puck or other member of soft material such as silicone. As shown in FIG. 19, the anus seal device 410 can be mounted to the patient with the valve 414 located adjacent to and/or extending through the patient's anus. Attachment structures such as adhesive strips 417 can be used to securely attached the anus seal device 410 to the patient's body. FIG. 19 illustrates a larger adhesive patch 419 that can conform to the patient's anatomy and enhance the attachment. By these approaches the valve 414 may not need to be inserted through the anus, and the adhesive patch 419, which can include an adhesive strip 421 around the valve 414, can provide a seal to inhibit insufflation gas from escaping during colonoscopy. The elongated portion 82 of the colonoscope 80 can be inserted into the patient's anus through the valve 414. In addition to inhibiting the escape of insufflation gas, anus seal device 410 allows the colonoscopist to move the colonoscope 80 through the patient's bowel and through the device during colonoscopy, in manners similar to those described above.

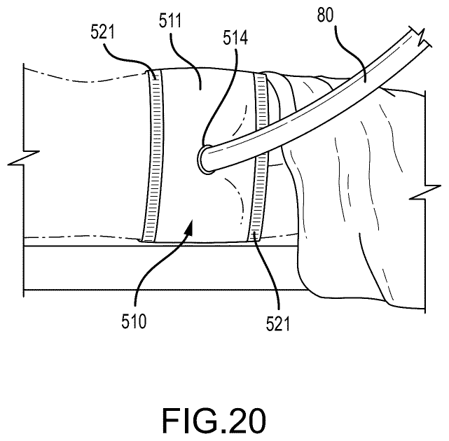

FIG. 20 illustrates an anus seal device 510 in accordance with embodiments. As shown, the anus seal device 510 includes a garment 511 that covers a portion of the patient's body that includes the anus, and a valve 514 at a location corresponding to the patient's anus. The garment 511 is a compression member in embodiments, and includes compression structures such as elastic portions 521 that seal all the openings in the garment. The illustrated embodiment of the garment 511 is an undershort, and the elastic portions 521 seal around the waist and legs of the patient. The fabric of the garment 511 can also be impervious to the insufflation gas. In effect, the garment 511 provides the seal functionality. The elongated portion 82 of the colonoscope 80 can be inserted into the patient's anus through the valve 514. In addition to inhibiting the escape of insufflation gas, anus seal device 510 allows the colonoscopist to move the colonoscope 80 through the patient's bowel and through the device during colonoscopy, in manners similar to those described above.

This disclosure is not meant to be read in a restrictive manner. For example, the terminology used in the application should be read broadly in the context of the meaning those in the field would attribute such terminology. Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” “downward,” “distal,” “proximal,” “leading” and “trailing” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Similarly, with term “substantially” may be used, in certain instances, to refer to an objective, result or outcome that includes the stated objective, result or outcome, and that also includes objectives, results or outcomes that are reasonably close to the stated objective, result or outcome. Objectives, results or outcomes that are reasonably close to the stated objective, result or outcome may deviate from the stated objective, result or outcome by a reasonably small amount as understood and readily ascertained by individuals having ordinary skill in the relevant arts. For example, objectives, results or outcomes that provide medically efficacious results may substantially meet the stated objective, result or outcome.

As used herein, “couple,” “attach” and “mount” mean join, connect, attach, adhere, affix, or bond, whether directly or indirectly, and whether permanently or temporarily.

For reference, the terms “circumference” “circumferentially” and “diameter” are not meant to require a circular cross-section or path (although are inclusive of a circular cross-section or path), and are instead to be understood broadly to reference an outer surface or dimension, the dimension between opposing sides of the outer surface, and paths or directions along such outer surfaces or dimensions.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

1. A device, comprising:
a seal member configured to be inserted into an anus of a patient and including a proximal end and a distal end, wherein the seal member defines a lumen extending through the seal member between the proximal end and the distal end and that is configured to allow a colonoscope to extend through the seal member and into a patient's bowel; and
a valve coupled to the seal member and configured allow the colonoscope to extend through the seal member and to inhibit insufflation gas from escaping the patient's bowl through the lumen.

2. The device of claim 1, wherein the seal member is a diametrically-expandable member.

3. The device of claim 2, wherein the seal member is a self-expandable member.

4. The device of claim 3, wherein at least portions of the seal member are configured to compress toward a reduced-diameter configuration when the device is inserted into the patient's anus.

5. The device of claim 3, further including a constraining member movable between a delivery position and an operative position with respect to the seal member, wherein:
when the constraining member is in the delivery position the constraining member constrains the seal member to a reduced-diameter delivery configuration; and
when the constraining member is in the operative position, the seal member expands.

6. The device of claim 2, wherein the seal member comprises an expandable balloon.

7. The device of claim 2, wherein the seal member is configured to engage at least portions of the patient's anal canal.

8. The device of claim 7, wherein at least portions of the seal member are configured to be located in the patient's rectum.

9. The device of claim 2, further comprising a hub including a tubular portion including a proximal end and a distal end extending through at least portions of the lumen of the seal member.

10. The device of claim 9, wherein the valve is coupled to the hub.

11. The device of claim 10, wherein the hub includes a base portion that extends from the proximal end of the seal member.

12. The device of claim 11, wherein at least portions of the valve are coupled to the base portion of the hub.

13. The device of claim 9, wherein at least portions of the tubular portion of the hub include an inner diameter tapering in a reducing-diameter configuration with increasing distance from the proximal end.

14. The device of claim 9, wherein the tubular portion of the hub includes one or more longitudinally-extending slots.

15. The device of claim 14, wherein:
at least portions of the tubular portion of the hub include an outer diameter tapering in a reducing-diameter configuration with increasing distance from the proximal end; and
at least portions of the lumen of the seal member include an inner diameter tapering in a reducing-diameter configuration with increasing distance from the proximal end, and the at least portions of the lumen of the seal member mate with the at least portions of the tubular portion of the hub.

16. The device of claim 9, wherein the distal end of the tubular portion of the hub does not extend to the distal end of the seal member.

17. The device of claim 2, wherein the seal member comprises a distal portion including an outer diameter that tapers between a first diameter and a second diameter that is less than the first diameter with increasing distance from the proximal end.

18. The device of claim 17, wherein the seal member comprises a proximal portion including an outer diameter that tapers between a third diameter that is greater than the first diameter and a fourth diameter that is less than the first diameter with increasing distance from the proximal end.

19. The device of claim 2, wherein the device is configured to open along its length to allow the device to be inserted from its side onto a colonoscope.

20. A method for performing colonography, comprising:
inserting a colonoscope through a valve in a lumen of an anal seal member;
inserting the colonoscope into an anus of a patient; and
insufflating the patient's bowel with gas through the colonoscope.

21. The method of claim 20, further comprising releasably attaching the anal seal member to a portion of the colonoscope not inserted into the patient's anus.

22. The method of claim 21, further comprising:
moving the anal seal member along the colonoscope from its releasably attached position, and inserting the anal seal member in the patient's anus; and
insufflating the patient's bowel with gas when the anal seal member is inserted in the patient's bowel.

23. The method of claim 20, further comprising:
inserting the anal seal member in the patient's anus; and
insufflating the patient's bowel with gas when the anal seal member is inserted in the patient's bowel.
